(19) United States
(12) Patent Application Publication
(10) Pub. No.: US 2012/0155607 A1
(43) Pub. Date: Jun. 21, 2012

(54) BELT-TYPE TABLE AND CT APPARATUS AND A METHOD FOR OBTAINING CALIBRATION DATA FOR A CT APPARATUS

(75) Inventors: Xiaoyan ZHANG, Beijing (CN); Hailiang LIU, Beijing (CN); Xuyong YANG, Beijing (CN); Xueli WANG, Beijing (CN)

(73) Assignee: GE MEDICAL SYSTEMS GLOBAL TECHNOLOGY COMPANY, LLC, Waukesha, WI (US)

(21) Appl. No.: 13/331,474

(22) Filed: Dec. 20, 2011

(30) Foreign Application Priority Data
Dec. 20, 2010 (CN) 201010615622.7

(51) Int. Cl. A61B 6/03; A61B 6/04

(52) U.S. Cl. 378/19; 5/601; 378/20

(57) ABSTRACT

A belt table is provided. The belt table includes a cradle, and a movement driver set configured to drive the cradle to move, wherein the cradle and the movement driver set are configured to cause at least one of low attenuation and nearly zero attenuation to X-rays along a scan plane.

### CROSS REFERENCE TO RELATED APPLICATIONS

This application claims the benefit of Chinese Patent Application No. 201010615622.7 filed Dec. 20, 2010, which is hereby incorporated by reference in its entirety.

### BACKGROUND OF THE INVENTION

The present invention generally relates to CT field, and in particular to a belt table, a CT apparatus, and a method for acquiring calibration data for the CT apparatus.

A CT apparatus has been widely used in the art to scan an object to obtain clear and distinct images of the scanned object. As known in the art, a typical CT apparatus as shown in FIG. 1 at least includes an X-ray tube 1, a detector 2 and a table 3. The X-ray tube 1 is used to emit X-rays, the detector 2 to receive the X-rays transmitted from the X-ray tube 1, the display console 50 to image the scanned object (e.g., a patient), and the table 3 to carry the scanned object. The table 3 includes a cradle 31 and a driver set. Generally, the X-ray tube 1 and the detector 2 are disposed on the opposing sides of the cradle 31 in the gantry.

U.S. Pat. No. 6,185,272B1 proposes an architecture for a CT scan system, which mainly includes a table for carry-on luggage in an airport. U.S. Pat. No. 7,072,434B1 also provides a carry-on baggage tomography scanning system, which is also used to scan luggage and articles.

Typically, the cradle 31 of the table 3 for use in the CT apparatus is made of a carbon fiber material, which is rather expensive and thus significantly increases the cost of the whole CT apparatus.

In addition, while the CT apparatus is scanning the object, the cradle 31 is required to slide the patient lying thereon. To this end, it is required that the sliding structure of the cradle 31 include rails, and sliding carriage, etc. This complicates the structure of the CT apparatus, and thus becomes another factor that contributes to the cost of the whole CT apparatus.

Furthermore, since the patient is lying on the cradle 31 while being scanned, the X-ray emitted from the X-ray tube 1 need to penetrate both the patient and the cradle 31 before reaching the detector 2. Therefore, the emitted X-rays are attenuated after travelling across the cradle 31. As a consequence, in order to obtain clear images, it is necessary to increase the X-ray dose to be applied to the patient. As known in the art, X-rays cause harm to the human body once they reach a certain dose.

Furthermore, the cradles of the existing tables have a cantilever beam structure in the direction across the scan plane. Consequently, there are chances that the cradle may sag when it continuously slides in the direction across the scan plane. Accordingly, different parts of the body being imaged become saggy in the image as well, and the patient body is no longer located at the clearest position on the image for a full scan range.

### SUMMARY OF INVENTION

The embodiments described herein provide a belt table having a safer and simpler structure, a CT apparatus, and a method for obtaining calibration data for the CT apparatus.

In one aspect, a belt table including a cradle and a movement driver set to drive the cradle to move is provided, wherein the cradle and the movement driver set cause low attenuation or nearly zero attenuation to X-rays along a scan plane.

In one embodiment, the movement driver set includes a driving part and a driven part, and a gap being arranged between the driving part and the driven part and configured to allow the scan plane to pass therethrough.

In one embodiment, the driving part includes an active roller, a passive roller, and a belt running over the active roller and the passive roller.

In one embodiment, the driving part further includes a middle support for supporting the belt.

In one embodiment, the active roller and the passive roller have teeth, and the belt has, on the inside surface, teeth to engage with the teeth on the active roller and the passive roller, and wherein the teeth do not traverse the scan plane in a scanning process.

In one embodiment, the movement driver set includes an active roller, a passive roller, and a belt that enables co-movement of the active roller and the passive roller, the belt being arranged only on the side where it is in contact with the cradle.

In one embodiment, the movement driver set includes an active roller, a passive roller and a belt running over the active roller and the passive roller.

In one embodiment, the movement driver set includes a middle support for supporting the belt, the middle support having a gap which is configured to allow the scan plane to pass therethrough.

In one embodiment, the cradle and the belt have, on respective sides where they contact each other, a concave structure and a convex structure respectively which mate with each other.

In one embodiment, the cradle and the belt have, on respective sides where they contact each other, a male connection and a female connection respectively, which mate with each other.

In one embodiment, the active roller and the passive roller are both provided with a flange.

In one embodiment, the passive roller includes a back tensile force spring in the middle.

In one embodiment, the middle support is a set of rollers.

In one embodiment, the middle support is an array of rollers.

In one embodiment, the middle support is a square block.

In one embodiment, the middle support is provided with a coating thereon.

In another aspect, a CT apparatus including a belt table as outlined above is provided.

In still another aspect, a method for acquiring calibration data for a CT apparatus is provided, the method including rotating an X-ray tube to a position between 0° and 180° where there is a virtual line between the focus of the X-ray tube and each of centers of detectors to scan an object to obtain first scan data rotating an X-ray tube to a position between 180° and 360° where there is a virtual line between the focus of the X-ray tube and each of centers of detectors to scan the object to obtain second scan data and combining the first scan data and the second scan data.

In one embodiment, combining the first scan data and the second data includes eliminating data relating to a table included in the CT apparatus.

As compared with the prior art, the belt table, the CT apparatus including the belt table and the method for acquiring calibration data for the CT apparatus described herein achieve the following advantageous technical effects:

Firstly, since the movement driver set employed in the embodiments described herein causes low attenuation or nearly zero attenuation to the X-rays along the scan plane, the cradle of the table may be fabricated with a small thickness. Due to the lowered X-ray attenuation, less X-ray dose will be needed to produce the images at the same resolution, thereby being much safer to the patients.

Secondly, the table according to the embodiments described herein does not rely on devices such as rails and sliding carriages, thereby simplifying the structure and reducing the cost.

Finally, the cradle according to the embodiments described herein no longer forms a cantilever beam structure. Consequently, the cradle will not become saggy in the direction perpendicular to the scan plane, which in turn produces clearer images.

### BRIEF DESCRIPTION OF THE DRAWINGS

To facilitate a thorough understanding of the embodiments described herein, reference is made to the following figures:

FIG. 1 is a schematic diagram showing a typical CT apparatus;

FIG. 2A is a rear view of an exemplary belt table;

FIG. 2B is a front view of the belt table shown in FIG. 2A;

FIG. 2C shows a cross sectional view of the belt table shown in FIG. 2A;

FIG. 3 shows a driving part and a driven part of the belt table;

FIG. 4 is a schematic diagram showing an alternative belt table;

FIG. 5 is a schematic diagram showing yet another alternative belt table;

FIG. 6 shows the combination of a cradle and a belt of the table;

FIG. 7 shows the matching of the cradle and the belt with a Velcro tape;

FIG. 8 shows an accurate inside-tooth belt driver mechanism;

FIG. 9 shows rollers with a flange at both ends;

FIG. 10 shows a driver mechanism with a back tensile force spring;

FIG. 11 illustrates a row of long, support rollers;

FIG. 12 shows an array of small, support rollers;

FIG. 13 illustrates a support block or plate;

FIG. 14 illustrates the flow of a method for acquiring calibration data for a CT apparatus;

FIG. 15 shows a scan system when the X-ray tube is at zero degree;

FIG. 16 shows a scan system when the X-ray tube is at a first scan position;

FIG. 17 shows a scan system when the X-ray tube is at a second scan position;

FIG. 18 illustrates a profile of data obtained at the first scan position using the method for acquiring calibration data for a CT apparatus;

FIG. 19 illustrates a profile of data obtained at the second scan position using the method for acquiring calibration data for a CT apparatus; and

FIG. 20 illustrates a profile of combined data obtained at the first and the second scan positions using the method for acquiring calibration data for a CT apparatus.

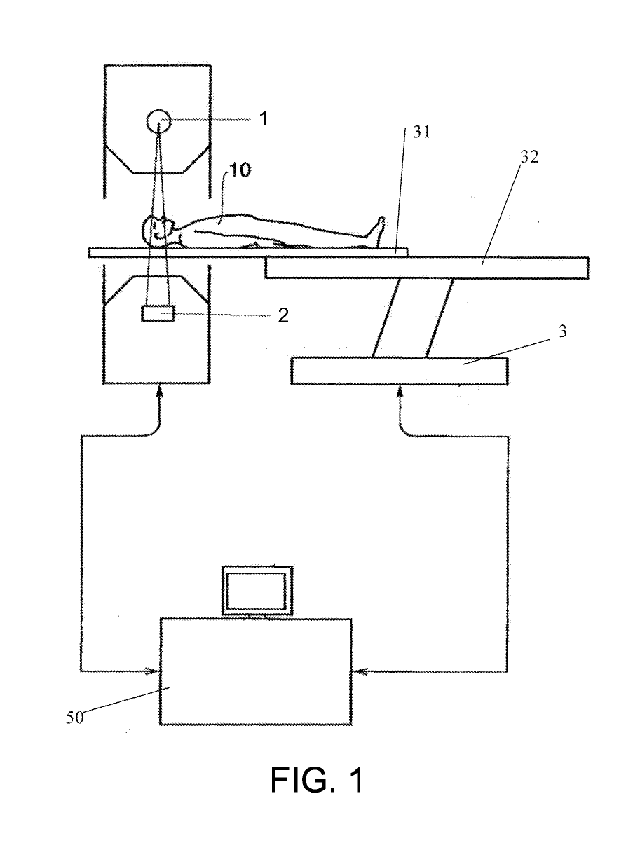

### DETAILED DESCRIPTION OF THE INVENTION

Exemplary embodiments are described in details hereunder. It should be understood that the present invention is not limited to these particular embodiments.

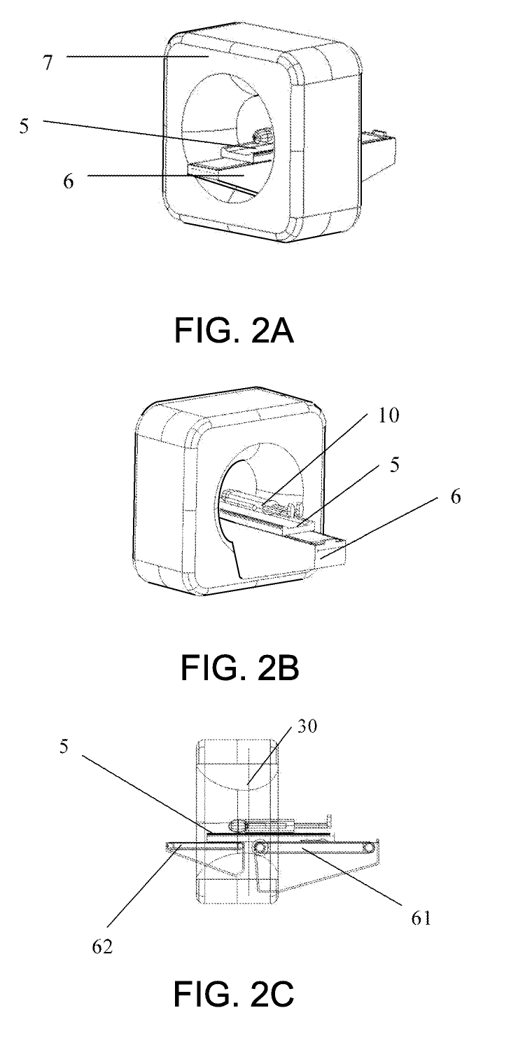

As shown in FIGS. 2A, 2B and 2C, the belt table includes a cradle 5 and a movement driver set 6 for driving the cradle 5 to move. The cradle 5 and the movement driver set 6 cause low attenuation or nearly zero attenuation to the X-rays along the scan plane 30.

Since the cradle 5 and the movement driver set 6 cause low attenuation or nearly zero attenuation to the X-rays along the scan plane, clear and distinct images may still be obtained using the present CT apparatus even when the tube 1 emits a relatively small dose of X-rays. Therefore, the belt table described herein is relatively safe for the scanned object.

As further shown in FIG. 2C, the movement driver set 6 includes a driving part 61 and a driven part 62, with a gap being arranged between the driving part 61 and the driven part 62. The driving part 61 drives the driven part 62 to move the cradle 5, wherein the scan plane 30 may pass through the gap, and wherein the gap is dimensioned to be slightly greater than the detection width of a detector.

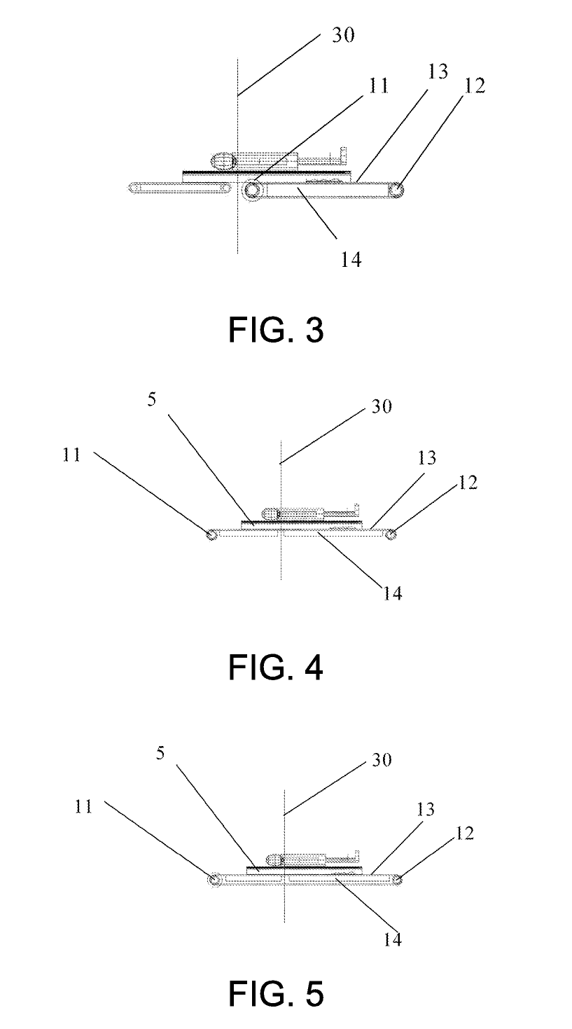

As shown in FIG. 3, the driving part 61 includes an active roller 11, a passive roller 12, and a belt 13 running over both the active roller 11 and the passive roller 12.

In addition, the driving part 61 further includes a middle support 14 for supporting the belt 13.

In one embodiment, as shown in FIG. 4, the movement driver set 6 includes an active roller 11, a passive roller 12 and a belt 13 that causes co-rotation of the active roller 11 and the passive roller 12. However, in this respect, the belt 13 is arranged only on the side where it is in contact with the cradle 5.

In the aforesaid embodiment, the belt 13 only runs over half of the active roller 11 and the passive roller 12. Therefore, only one layer of the belt 13 causes attenuation to the emitted X-rays.

FIG. 5 shows another embodiment, wherein the movement driver set 6 includes an active roller 11 and a passive roller 12 and a belt 13 that runs over the active roller 11 and the passive roller 12.

As shown in FIG. 5, since the belt 13 runs over the entire active roller 11 and passive roller 12, two layers of the belt 13 will cause attenuation to the emitted X-rays.

The belt 13 as shown in FIG. 4 and FIG. 5 may be made of materials that cause small attenuation to the X-rays.

In one embodiment, as shown in FIG. 8, both the active roller 11 and the passive roller 12 have teeth 21, and the belt 13 also has, on its inside surface, teeth that engage with those on the active roller 11 and the passive roller 12. Thereby, the embodiments described herein can provide a table with higher driving accuracy. Furthermore, the belt as shown in FIGS. 4 and 5 is provided with teeth on the sides but no teeth in the middle in order not to cause unfavorable effects to the image quality.

According to the embodiment as shown in FIGS. 4 and 5, the movement driver set 6 further includes a middle support 14 for supporting the belt 13. A gap is arranged where the middle support 14 intersects the scan plane 30, and is dimensioned to allow the scan plane to pass therethrough. Put it another way, the middle support 14 does not cause attenuation to the X-rays. As mentioned above, the gap is slightly wider than the detection width of the detector.

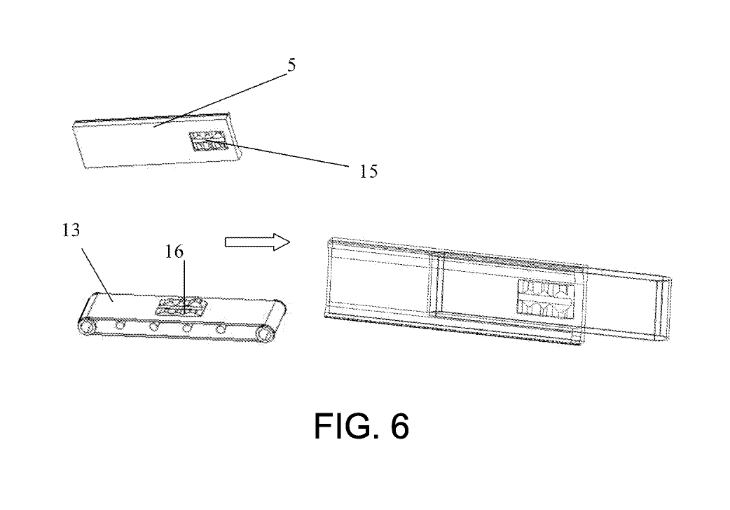

As shown in FIG. 6, the cradle 5 and the belt 13 are provided, on respective sides that contact each other, with a concave structure 15 and a convex structure 16 respectively, which mate with each other. The concave structure 15 and the convex structure 16 may cooperate to limit the movement of the cradle 5, such that there are no translations or rotations between the cradle 5 and the belt 13.

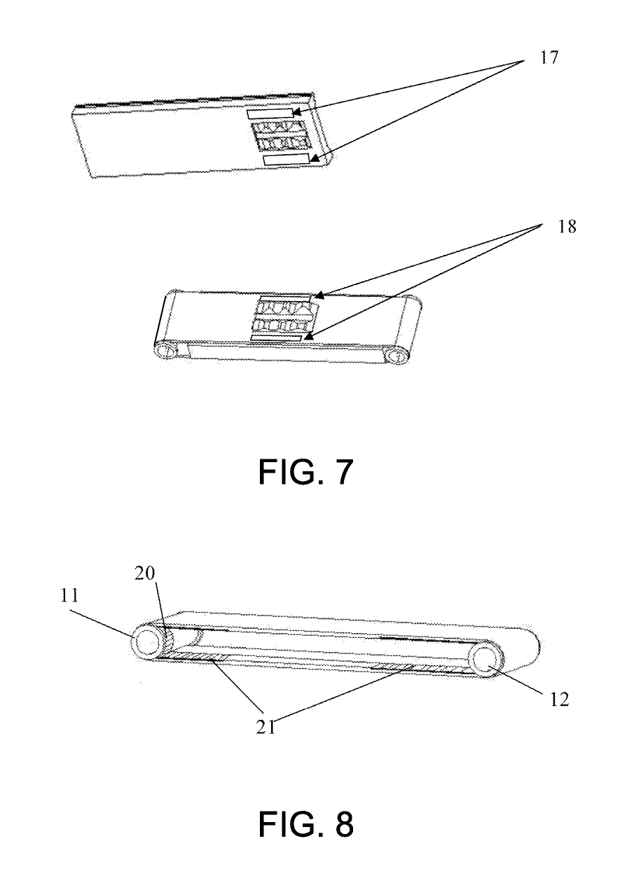

In addition, according to FIG. 7, on respective sides where the cradle 5 and the belt 13 contact each other are provided with a male connection 17 and a female connection 18 respectively, which may be secured to each other with a Velcro tape. Thereby, the cradle 5 may not be separated from the belt 13. In other words, the cradle 5 may be completely tied to the belt 13.

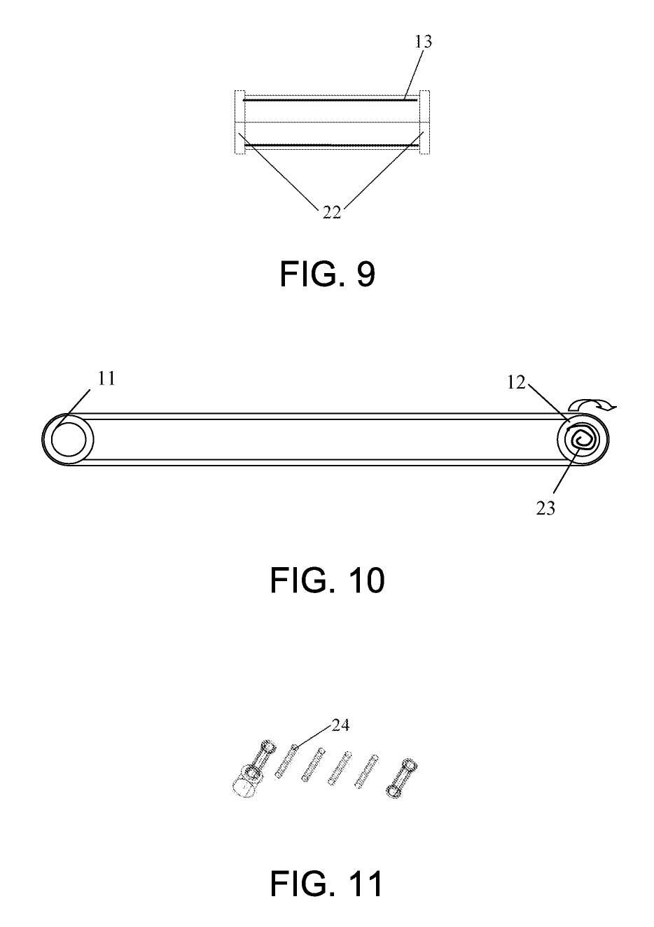

As shown in FIG. 9, both the active roller 11 and the passive roller 12 have a flange 22 thereon, which is capable of limiting the horizontal translation of the belt 13.

According to one embodiment, as shown in FIG. 10, the passive roller 12 is provided with a spring 23 in the middle, which provides a back tensile force for the belt such that the belt always remains in tension to reduce errors in the movement in the reversed direction.

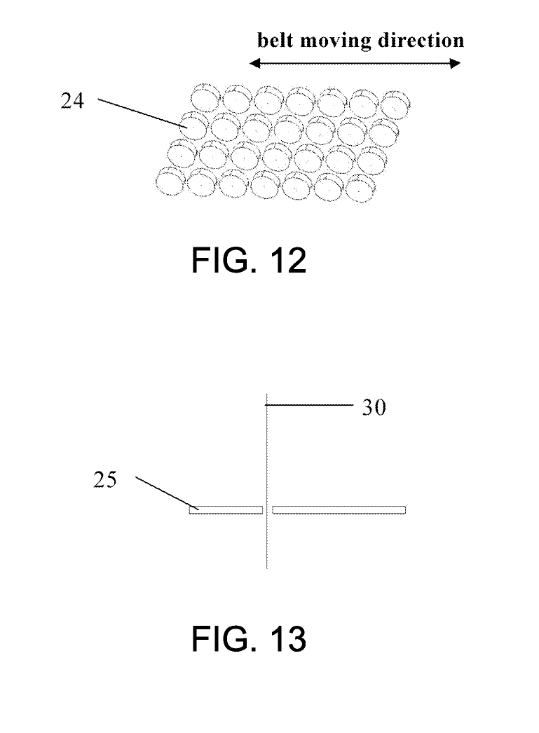

FIG. 11 shows that the middle support 14 may be configured as a row of small rollers 24. Alternatively, the middle support 14 may form an array of groups of small rollers 24, as shown in FIG. 12.

The middle support 14 may be formed into a square block 25 as shown in FIG. 13. In one embodiment, the square block 25 is provided with a coating which may provide a smooth and wear-resistant surface.

In addition, the belt table according may further include a position detector and controller (not shown) for detecting and controlling the position, the velocity, and the acceleration of the cradle.

In another aspect, a CT apparatus, which includes the foregoing belt table, is provided. As details of the belt table are discussed above, the belt table is not discussed in detail with reference to the CT apparatus.

Generally, periodic calibrations need to be conducted for a CT apparatus. The calibration processes may produce Aircal, G sin and G cos, BH, BIS, and CT# adjustment (CT data adjustment), etc. Acquisition of a complete set of scan data is the initial and utmost important matter for the calibration of a CT apparatus. The term “complete set of data” refers to data obtained upon scanning an air or water phantom. The existing technology generally requires removing the table from the CT apparatus before scanning the air or water phantom to obtain the complete set of scan data. It is well known that removal of the table could be rather troublesome. Therefore, it has been a major subject and concern in the art to obtain calibration data for CT apparatus without the need to remove the table.

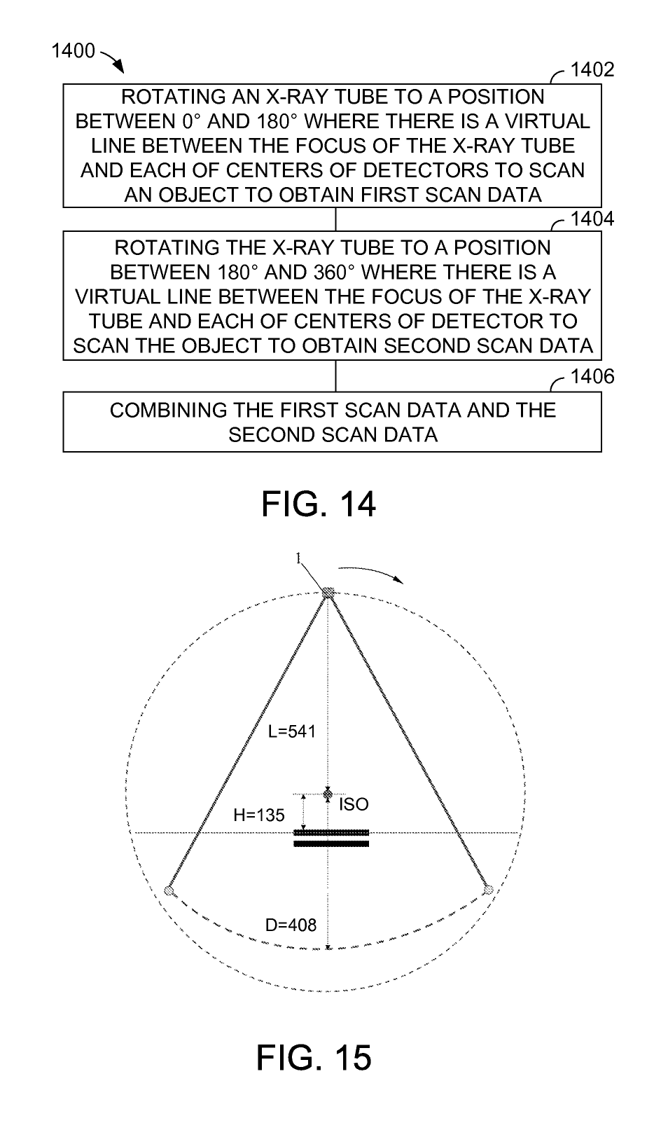

As shown in FIG. 14, a method 1400 for acquiring calibration data for a CT apparatus includes rotating 1402 an X-ray tube to a position between 0° and 180° where there is a virtual line between the focus of the X-ray tube and each of centers of detectors to scan an object to obtain first scan data, rotating 1404 the X-ray tube to a position between 180° and 360° where there is a virtual line between the focus of the X-ray tube and each of centers of detectors to scan the object to obtain second scan data, and combining 1406 the first scan data and the second scan data.

As such, the tube 1 is first rotated to a position between 0° and 180° where there is a virtual line between the focus of the X-ray tube and each of centers of detectors, and emits X-rays to the object to be scanned to obtain the first scan data. The position between 0° and 180° as mentioned is the position at which there could be a virtual line between the focus of the X-ray tube and each of centers of detectors. Assuming these lines are L1, L2, . . . , Ln, when a certain line of these lines overlaps the center line of the table in Y direction (i.e., vertical direction), a stationary (non-rotary) scan is conducted at the position concerned where said overlapping occurs. After the first scan data is obtained, the X-ray tube 1 continues to be rotated to a position between 180° and 360° where there is a virtual line between the focus of the X-ray tube and each of centers of detectors, and emits X-rays to the object to be scanned to obtain the second scan data. The position between 180° and 360° as mentioned is the position at which there could be a virtual line between the focus of the X-ray tube and each of centers of detectors. Assuming these lines are L1, L2, . . . , Ln, when a certain line of these lines overlaps the center line of the table in Y direction (i.e., vertical direction), a stationary (non-rotary) scan is conducted at the position concerned where said overlapping occurs. It can be seen that both the first scan data and the second scan data include the data related to the table body, but the data related to the table body is within different channels. Finally, the first and second scan data may be combined to form a complete set of scan data.

Of course, persons skilled in the art would understand that a selectable area may be also an angular position located within the angular range deviating from the aforesaid overlapping position by a certain angle, for example, 20°.

Combining the first scan data and the second scan data includes eliminating the data related to the table body.

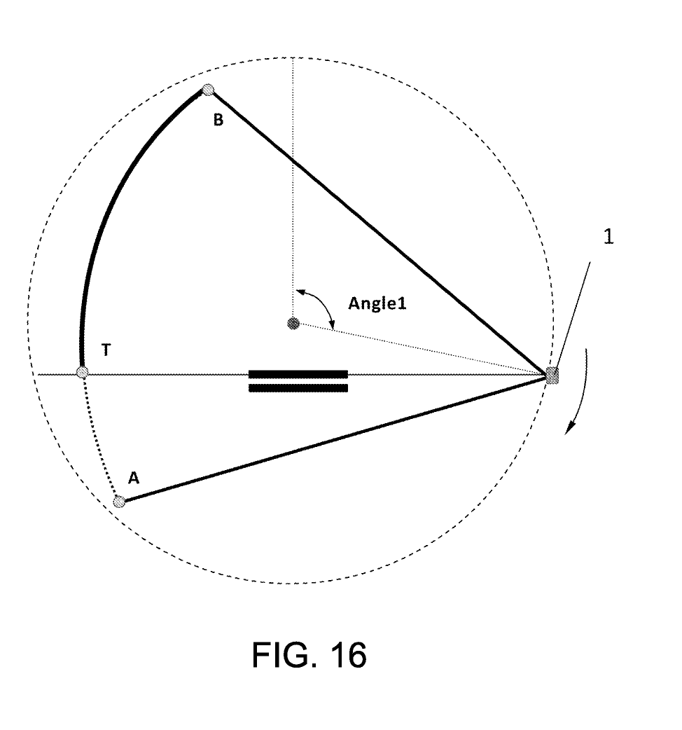

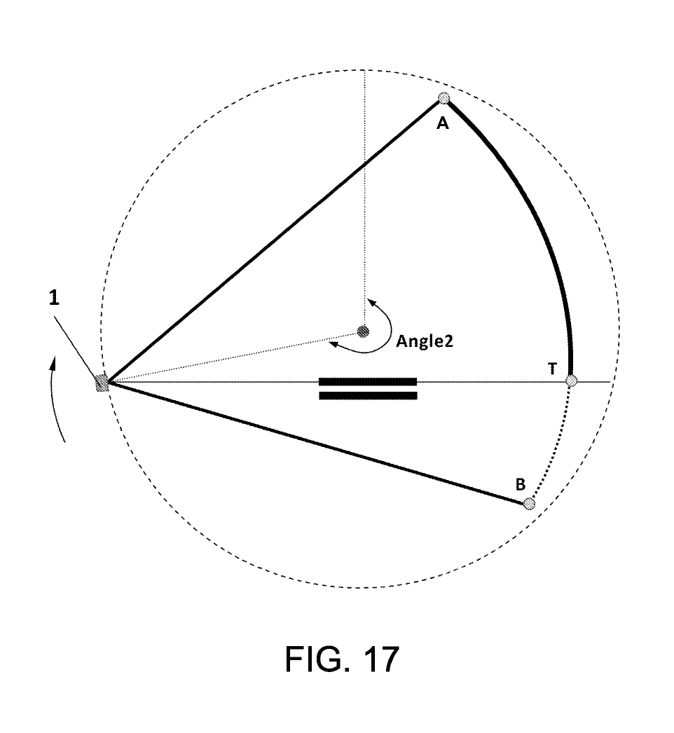

As described above, the method for acquiring calibration data for a CT apparatus includes rotating the X-ray tube 1 to the positions as respectively shown in FIGS. 16 and 17, and emitting X-rays to carry out stationary (non-rotary) scan to obtain first scan data and second scan data.

For example, FIG. 15 illustrates the gantry with the X-ray tube 1 being at 0°. As shown, “L” represents the distance between the X-ray tube and the virtual center of rotation (ISO), “H” represents the distance between the table body and the virtual center of rotation (ISO), and “D” represents the distance between the detector and the virtual center of rotation (ISO).

In the embodiment as shown in FIG. 16, the X-ray tube and the detector are arranged at the first scan position. As shown, “A” and “B” represent both ends of the detector, and T represents the channel position where the detector is capable of receiving signals from the table. In this embodiment, the X-ray tube 1 is rotated to an angle defined by an equation: Angle1=PI/2+Actan(H/L), where “PI” represents π, “Actan” represents a tangent function, “H” represents the distance between the table and the virtual center of rotation (ISO), and “L” represents the distance between the X-ray tube and the virtual center of rotation (ISO).

FIG. 17 illustrates the embodiment in which the X-ray tube and the detector are arranged at the second scan position. As shown, “A” and “B” represent both ends of the detector, and “T” represents the channel position where the detector is capable of receiving signals from the table. In this embodiment, the X-ray tube 1 is rotated to an angle defined by an equation: Angle2=PI*3/2−Actan(H/L). For example, as shown in FIG. 15, L is equal to 541, and H is equal to 135. Consequently, Angle1 is equal to 104° and Angle2 is equal to 256°.

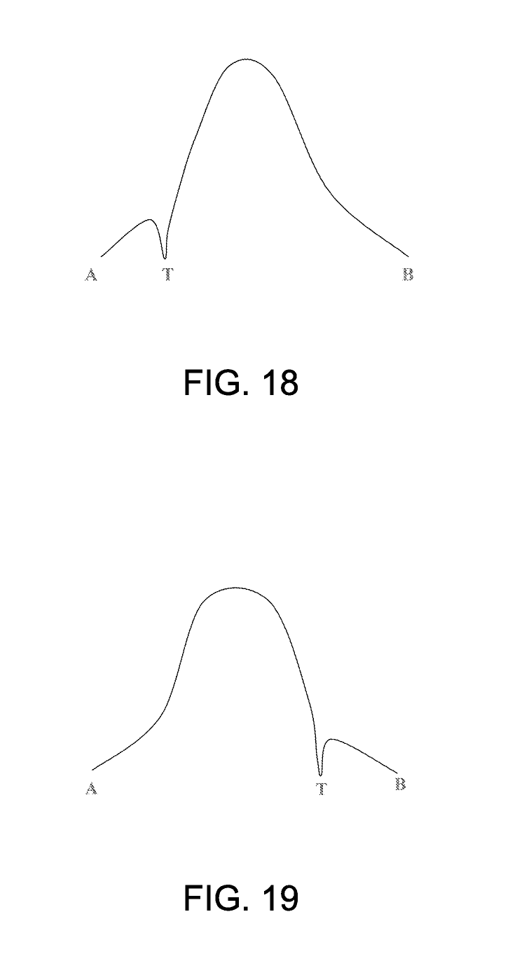

The data obtained at the first scan position may have a profile substantially as shown in FIG. 18, and the data obtained at the second scan position may have a profile substantially as shown in FIG. 19. The data of FIGS. 18 and 19 have been subjected to common processing, including dark current correction, reference channel correction and de-ganging.

The combination of the first scan data and the second scan data is introduced here below.

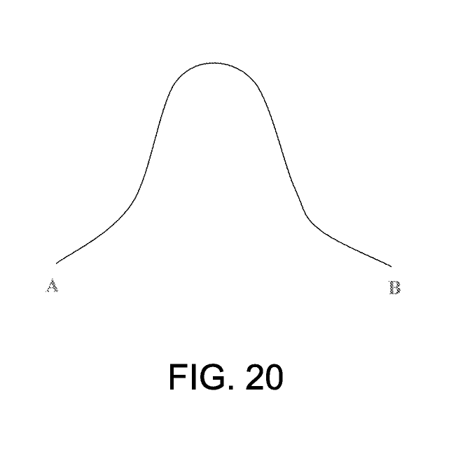

A region in proximity to position T is selected in both FIG. 18 and FIG. 19. Position T is the channel nearest to the center line of the table in Y direction. As described above, channel data in the selected region, which includes data of the table body itself, is excluded or eliminated (for example, the relevant data is set to zero). Similar operations are carried out with respect to the scan data obtained at both the first position and the second position. Subsequently, the data with the table data having been eliminated is summed up channel by channel. Thereafter, the sum of the data excluding the table data (e.g., the non-zero data) at the first and second scan positions is divided by 2, while the rest of the data remains unchanged. So, the summed result may have the profile as shown in FIG. 20.

It is apparent from above that the movement driver set supports the cradle at both ends. Thereby, the embodiments described herein have significantly less requirements on the rigidness and strength of the cradle. Materials of low density and low strength may be used for the cradle, for example, a foamed plastic, industrial organic glass, and density plate, which have low X-ray attenuation. On the other hand, since the distance between the supporting locations where the movement driver set supports the cradle at both ends is small, the cradle may be designed with a smaller thickness. The cradle with the lowered thickness reduces the attenuation of X-rays along the scan plane, which in turn requires less X-ray dose to produce the images of the same resolution.

Finally, the method for acquiring calibration data for a CT apparatus described herein is capable of obtaining a complete set of calibration data without removing the table, which significantly increases efficiency.

It is to be understood, however, that even though numerous characteristics and advantages of the present invention have been set forth in the foregoing description in the combination of the accompanying drawings, the disclosure is illustrative only, and changes, modifications and equivalent substitutions may be made by skilled persons in the art without departing from the invention herein. These changes, modifications and equivalent substitutions fall within the spirit and scope of the present invention defined by the appended claims.

1. A belt table comprising:
a cradle; and
a movement driver set configured to drive the cradle to move, wherein the cradle and the movement driver set are configured to cause at least one of low attenuation and nearly zero attenuation to X-rays along a scan plane.

2. The belt table according to claim 1, wherein the movement driver set comprises a driving part, a driven part, and a gap arranged between the driving part and the driven part and configured to allow the scan plane to pass therethrough.

3. The belt table according to claim 2, wherein the driving part comprises an active roller, a passive roller, and a belt running over the active roller and the passive roller.

4. The belt table according to claim 3, wherein the driving part further comprises a middle support configured to support the belt.

5. The belt table according to claim 4, wherein the active roller and the passive roller comprise teeth, and an inside surface of the belt comprises teeth configured to engage the teeth on the active roller and the passive roller, wherein the teeth do not traverse the scan plane during a scanning process.

6. The belt table according to claim 1, wherein the movement driver set comprises an active roller, a passive roller, and a belt configured to drive co-movement of the active roller and the passive roller, the belt arranged only on a side where the belt contacts the cradle.

7. The belt table according to claim 1, wherein the movement driver set comprises an active roller, a passive roller and a belt running over the active roller and the passive roller.

8. The belt table according to claim 7, wherein the active roller and the passive roller comprise teeth, and an inside surface of the belt comprises teeth configured to engage the teeth on the active roller and the passive roller, wherein the teeth do not traverse the scan plane during a scanning process.

9. The belt table according to claim 6, wherein the movement driver set comprises a middle support configured to support the belt, the middle support comprising a gap configured to allow the scan plane to pass therethrough.

10. The belt table according to claim 3, wherein a side of the cradle that contacts the belt comprises a concave structure, and a side of the belt that contacts the cradle comprises a convex structure, the concave and convex structures configured to mate with each other.

11. The belt table according to claim 10, wherein a side of the cradle that contacts the belt comprises a male connection, and a side of the belt that contacts the cradle comprises a female connection, the male and female connection configured to mate with each other.

12. The belt table according to claim 11, wherein the active roller and the passive roller both comprise a flange.

13. The belt table according to claim 12, wherein the passive roller comprises a back tensile force spring in a middle of the passive roller.

14. The belt table according to claim 10, wherein the middle support comprises a set of rollers.

15. The belt table according to claim 10, wherein the middle support comprises an array of rollers.

16. The belt table according to claim 10, wherein the middle support comprises a square block.

17. The belt table according to claim 16, wherein the middle support comprises a coating thereon.

18. A CT apparatus comprising:
a belt table comprising:
a cradle; and
a movement driver set configured to drive the cradle to move, wherein the cradle and the movement driver set are configured to cause at least one of low attenuation and nearly zero attenuation to X-rays along a scan plane.

19. A method of acquiring calibration data for a CT apparatus, comprising:
rotating an X-ray tube to a position between 0° and 180° such that there is a virtual line between a focus of the X-ray tube and each of centers of a plurality of detectors, wherein the X-ray tube is rotated to scan an object to obtain first scan data; rotating the X-ray tube to a position between 180° and 360° such that there is a virtual line between the focus of the X-ray tube and each of centers of the plurality of detectors, wherein the X-ray tube is rotated to scan the object to obtain second scan data; and
combining the first scan data and the second scan data.

20. The method according to claim 19, wherein combining the first scan data and the second data comprises eliminating data relating to a table body included in the CT apparatus.
